(19) United States
(12) Patent Application Publication
(10) Pub. No.: US 2010/0311040 A1
(43) Pub. Date: Dec. 9, 2010

(54) Dna Fragments, Primers, Kits, and Methods for Amplification the Detection and Identification of Clinically Relevant Candida Species

(75) Inventors: Agostinho Alberico Rodrigues Carvalho, Braga (PT); Fernando Jose Dos Santos Rodrigues, Braga (PT)

Correspondence Address: SUGHRUE MION, PLLC, 2100 PENNSYLVANIA AVENUE, N.W., SUITE 800, WASHINGTON, DC, 20037, US

(73) Assignee: Universidade Do Minho, Braga (PT)

(21) Appl. No.: 11/914,845

(22) PCT Filed: May 16, 2006
(86) PCT No.: PCT/IB06/51546

§ 371 Date: Nov. 19, 2007

(30) Foreign Application Priority Data
May 17, 2005 (PT) 103277

(51) Int. Cl. C12Q 1/68; C07H 21/00; C12P 19/34

(52) U.S. Cl. 435/6; 536/23.74; 536/24.33; 435/91.2

(57) ABSTRACT

The present invention describes a novel molecular method based on the polymerase chain reaction (PCR) in a multiplex variant in order to detect and identify Candida species with clinical relevance, namely C. albicans, C. glabrata, C. krusei, C. parapsilosis, C. tropicalis, C. guilliermondii, C. lusitaniae e C. dubliniensis. The strategy uses the existence of sequences, whether conserved or variable, in fungal ribosomal genes and in the use of a combination of universal primers, specific for fungi, and internal primers, specific for each one of the Candida species (FIG. 1). In this sense, two fragments from the internal transcribed spacer (ITS) regions of the ribosomal RNA (rRNA) are amplified by multiplex PCR, allowing the easy identification of the Candida species in question. This methodology allows a rapid, effective and low-cost identification of Candida species with clinical relevance, bearing several advantages over the currently available diagnostic methods.

### SCOPE OF THE INVENTION

The described invention is included in the detection and identification of clinically important fungi. More particularly, this inventions is related with the identification of Candida species with clinical relevance, namely C. albicans, C. glabrata, C. krusei, C. parapsilosis, C. tropicalis, C. guilliermondii, C. lusitaniae and C. dubliniensis.

### BACKGROUND OF THE INVENTION

In the last years, changes in the epidemiological profile of infectious diseases have been observed characterized with the increase of fungal infections when compared to bacterial infections, clearly proven by a 400% increase in fungemias from 1990 to 2000 (Kao et al., Clin. Infect. Dis. 29:1164-70 (1999)), with the consequent elevated cost for the public health system (Edmond et al., Clin. Infect. Dis. 29:239-244 (1999)).

Invasive fungal infections, namely candidemia, assume particular relevance in nosocomial infections (Jarvis et al., Clin. Infect. Dis. 20:1526-30 (1995); Fridkin et al., Clin. Microbiol. Rev. 9:499-511 (1996)).

Although the C. albicans species is still the most common etiological agent in fungal infections, the frequency of other species, including C. glabrata, C. tropicalis, C. parapsilosis, C. krusei, C. guilliermondii, C. lusitaniae and C. dubliniensis, has been increasing in an expressive form (Pfaller et al., J. Clin. Microbiol. 40:3551-57 (2002); Diekema et al., J. Clin. Microbiol. 40:1298-302 (2002)). Thus, considering that this kind of infections presents high morbidity and mortality rates, a precise and fast diagnostic of the infectious agent assumes increasing relevance in clinical practice. On the other hand, and since susceptibility to antifungal drugs varies among Candida species, the identification of the species involved in the infection is fundamental to a proper antifungal therapy (Nguyen et al., Am. J. Med. 100:617-23 (1996)). In this sense, a rapid and efficient diagnostic method would benefit not only the patients, but also health institutions, eliminating the risk of inadequate therapies and prolonged hospitalization periods, diminishing the costs associated to the treatment of this kind of infections.

The laboratory diagnosis and treatment of fungal infections are usually problematic and are still a great challenge both to clinicians as well as to microbiologists. The fungi are difficult to cultivate from samples such as blood or urine and taking into account their ubiquity, a positive culture presents a limited clinical value due to the elevated probability of contamination. Nowadays, the standard method used to diagnose candidemia consists in retrieving the microorganism by hemoculture (Kiehn et al., J. Clin. Microbiol. 14:681-83 (1981); Roberts et al., J. Clin. Microbiol. 1:309-10 (1975)). The isolation of Candida from blood is a highly predictive fact of invasive infections, although the success rate of cultivation is inferior to 20% in patients with candidemia. In addition, it is necessary to take into account the time necessary to cultivate and identify the yeast, a generally time-consuming procedure.

The presence of specific antibodies against Candida in the serum is also used as a diagnostic criterion by the determination of the titre of antibodies present in the serum. Nevertheless, the sensitivity of this methodology is very low (usually inferior to 50%), since the patients that are immunocompromised have difficulty to generate adequate immune responses. The flaws, from both the cultural methods as well as those from the antibody detection, resulted in that the attention of researchers was focussed in tests that detected antigens or fungal metabolites in body fluids. A major problem of this technique is the transient nature of the antigens in the serum, whereby the sensibility is generally much reduced for most of the antigen tests. In addition, none of the afore-mentioned methods allow the identification of the fungus to the species level, necessary for the effective treatment of fungal infections.

Recently, several techniques based on PCR reactions have been used to identify fungus to the species level. Buchman et al. were the first to describe the use of PCR for identification of C. albicans in clinical samples (Buchman et al., Surgery 108:338-47 (1990)). These researchers used PCR to amplify part of a specific gene encoding cytochrome lanosterol 14-alpha demethylase. The predicted PCR product was approximately 240 bp, however unexplained amplification patterns were observed in several clinical samples containing DNA from C. albicans. In addition, the set of primers used by Buchman et al. amplified DNA from species other than C. albicans, resulting in PCR products with the ‘predicted’ size of 240 bp.

The U.S. Pat. No. 6,017,699 describes a set of primers that, when used in a PCR reaction, allow to amplify and speciate DNA from 5 clinically relevant Candida species. The amplified PCR products can be used to create specific DNA probes that can also allow the detection and identification of 5 species of Candida.

Nowadays, ribosomal genes are common targets in the design of strategies for the identification of fungi. In spite of the elevated level of conservation of the mature sequences of rRNA, the spacer transcribed and non-transcribed sequences are generally poorly conserved and, in this sense, they can potentially be used as target sequences for the detection of evolutive differences. Fungal rRNA genes are organized in units, with each one encoding three mature subunits: 18S, 5.8S and 28S. These subunits are separated by two internal transcribed spacer regions, ITS-1 and ITS-2, of approximately 300 bp. The highly variable sequences of the internal transcribed spacer regions ITS-1 and ITS-2, flanked by the relatively conserved coding regions of the nuclear rRNA genes 18S, 5.8S and 28S, have been used in various formats in PCR-based identification of yeast from the Candida genera.

The WO9323568 discloses diagnosis methods of fungal infections through the detection of distinct regions of the pathogenic fungus genome, such as the Candida genera, including the 5S region of rRNA and the ITS regions of rRNA. In addition, fungal detection methods as from these regions are described, namely by the use of DNA probes or specific primers for these regions.

The U.S. Pat. No. 5,426,027 describes isolated nucleic acids consisting essentially of specific nucleotide sequences of the ITS-2 region from C. albicans, C. parapsilosis, C. tropicalis, C. glabrata and C. krusei and, additionally, a method for the diagnostic of candidemia consisting of the following steps: blood collection, lysis of Candida cells and DNA precipitation, amplification of precipitated DNA with universal fungal primers derived from the ITS regions and detection of Candida DNA amplified by probes that selectively hybridize, thus indicating the presence of candidemia.

Williams et al. (1995) demonstrated the possibility to identify Candida species through the PCR amplification and analysis of the DNA fragments resulting from the use of restriction enzymes in the amplified ITS regions (Williams et al., J. Clin. Microbiol. 33:2476-79 (1995)). Fujita et al. described non-isotopic DNA probes labeled with digoxigenin for the ITS-2 region of different Candida species (Fujita et al., J. Clin. Microbiol. 33:962-67 (1995)). These probes were used in a plate microtitration method to rapidly detect and identify genomic DNA from C. albicans in blood. In turn, Shin et al. (1999) described the detection and identification of three different species of Candida in a single reaction, using amplification with the universal primers ITS3 and ITS4 and hybridization with probes for the ITS-2 region (Shin et al., J. Clin. Microbiol. 35:1454-59 (1997)). Finally, Chang et al. (2001) applied a multiplex PCR method in the identification of Candida in positive hemocultures, although it was only possible to identify a maximum of six species, requiring two independent multiplex PCR reactions (Chang et al., J. Clin. Microbiol. 39:3466-71 (2001)).

Although the molecular methods available present an increased specificity, they are generally costly or compel sophisticated technology not readily available or not easily implementable in diagnostic laboratories. In addition, conventional methods of identification do not allow distinguishing and identifying two or more Candida species present in multiple infections, a fact occurring with some frequency and that can affect the antifungal therapy. On the other hand, species that share many biochemical characteristics are easily mistaken by conventional methods, a situation occurring with C. albicans and C. dubliniensis (Bikandi et al., J. Clin. Microbiol. 36:2428-33 (1998)). Finally, most of the genotypical methods of identification are based on the use of purified DNA of the species to identify, which invariably leads to the need of implementing time-consuming methods of DNA isolation. As a consequence, the development of a rapid, effective, low-cost and easily applicable method is of great importance, allowing the implementation of effective therapeutic regimes and the monitoring of the patients' progression.

### SUMMARY OF THE INVENTION

The present invention responds to the existing need for a rapid, precise and cost-effective method compared to those available nowadays, whether in clinical terms or in the field of research. Thus, a method based on multiplex PCR is described for the detection and identification of Candida species with relevant interest in clinical practice. More particularly, the used strategy uses essentially three factors: (i) the elevated number of copies from the rRNA genes (about 100 copies per genome), (ii) the differences regarding the sizes of the ITS regions and (iii) the elevated variability of these region sequences among the different species of Candida. Thus, this technique is based on the amplification of DNA fragments specific of the internal transcribed spacer regions 1 (ITS-1) and 2 (ITS-2) by multiplex PCR. The methodology uses the combination of two universal primers and seven specific primers for each one of the Candida species studied, in a single PCR reaction, originating two fragments of different sizes for each species, with the exception of C. glabrata (FIG. 1; FIG. 2A). In this last case, the size of the ITS-1 and ITS-2 regions, including the rRNA coding region 5.8S, is sufficient to discriminate C. glabrata from the other species. The presented method allows the detection and differentiation of Candida species in a rapid, precise and specific manner, being a useful tool in the clinical diagnosis of fungal infections. In addition, this methodology can be used in the monitorization of fungal infections and to guide an appropriate antifungal therapy. The strategy described can also be applied in research laboratories in order to study the different fungal species and its phylogenetic proximity. The Candida species detected and identified by the method of the invention include the species with higher clinical relevance, namely C. albicans, C. glabrata, C. krusei, C. parapsilosis, C. tropicalis, C. guilliermondii, C. lusitaniae and C. dubliniensis.

The simplicity and efficiency of this method are also shown by other characteristics displayed by the described invention. The designed strategy allows the use of whole cells directly in the PCR reaction, bypassing the time-consuming and laborious steps necessary for the DNA isolation and reducing the time required for the identification (FIG. 2B). Additionally, a PCR-based method has the possibility to detect both viable as well as dead cells, increasing many fold the target sequence to amplify, being advantageous when compared to cultural methods.

On the other hand, the fact that of also being possible to detect and identify yeasts present in peripheral blood assumes great clinical relevance, since it not necessary to wait for the positivity of the hemoculture thus bypassing the usually large periods of time that the subculturing method imply. In fact, with the method proposed in the present invention, 1.5 hours are needed for the disruption of blood cells and Candida DNA isolation, 2 hours for the DNA amplification by multiplex PCR and 1 hour for agarose gel electrophoresis and visualization of the results. In this way, the species can be identified in about 4.5 hours, in contrast to the phenotypical methods that can take several days. The minimum number of Candida cells detected by this methodology is about 800 CFU/ml, an acceptable value when taking into account that when a hemoculture becomes positive, the number of cells present usually is about 10 CFU/ml. Nevertheless, this value could be further diminished by purification of an isolated DNA with the objective of eliminating possible PCR inhibitory factors (Panaccio et al., Nucleic Acid Res. 19:1151 (1991); Maaroufi et al., J. Clin. Microbiol. 42:3159-63 (2004)).

In addition, the involved etiological agents in polyfungal Candida infections are not identified separately by conventional methods, including the commercial identification systems, while the herein described method allows not only the identification of Candida species separately, but also in combinations with each other (FIG. 2C). In fact, this method has the capacity to detect polyfungal infections in a discriminatory fashion from a hemoculture, contrarily to what occurs with the conventional methods of isolation by subculture, in which the ratio of the proliferation of the different cellular types has a strong impact in the identification. In the particular case of mixed microbial cultures (simultaneous fungal and bacterial growth), no detectable PCR products using DNA from Escherichia coli, Staphylococcus aureus, Pseudomonas aeruginosa and Bacillus subtilis were found, although the Candida species were identified without any kind of interference.

Also in economical terms, this strategy reveals extremely attractive, when taking into account that most of the expenses related to the components of the PCR mixture, not being necessary DNA probes or restriction enzymes of elevated cost. Finally, one the major advantages that this technology presents is the reproducibility of the technique when performed by different persons and using different machines, what per se facilitates extremely its implementation in diagnostic laboratories. Additionally, its execution requires just common equipment generally in use in most of the clinical laboratories and if not, easily implemented. Altogether, the features presented herein by this new technique together with the easiness of interpretation of the results point to a highly advantageous applicability concerning the identification of the etiological agent in Candida infections, both in the clinical practice as well as in epidemiological studies.

### BRIEF DESCRIPTION OF THE FIGURES

Table I describes the universal and specific primers used for each Candida species in the presented multiplex PCR methodology, the respective nucleotide sequence and the predicted size of the obtained fragments by agarose gel electrophoresis.

FIG. 1 illustrates the presented multiplex PCR strategy. Particularly, FIG. 1 represents the organization of the fungal ribosomal genes and the indication of the universal (UNI1 and UNI2) and specific (Calb, Cgla, Ckru, Cpar, Ctro, Cgui, Clus and Cdub) primer targets.

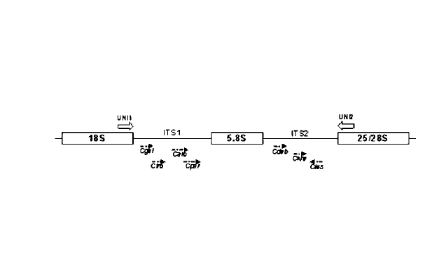

FIG. 2 (2A, 2B and

2C)

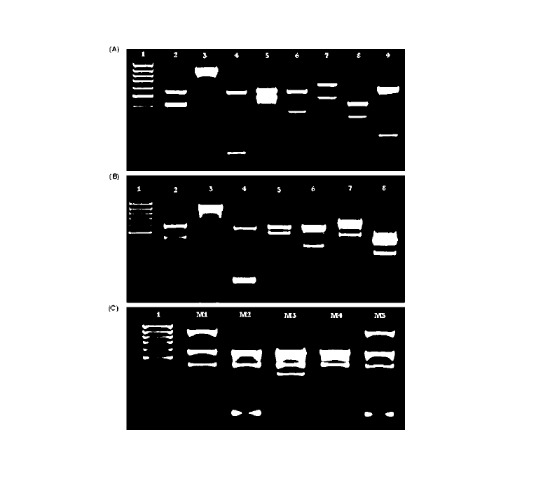

shows the experimental results obtained by agarose gel electrophoresis after multiplex PCR amplification. More specifically, FIG. 2A represents the amplification of isolated Candida DNA, FIG. 2B represents the amplification using whole cells of Candida and finally, FIG.

2C

shows the result obtained when Candida polyfungal cultures are used (lane 1—100 bp DNA ladder; lane 2—C. albicans; lane 3—C. glabrata; lane 4—C. krusei; lane 5—C. parapsilosis; lane 6—C. tropicalis; lane 7—C. guilliermondii; lane 8—C. lusitaniae; lane 9 C. dublininiensis; lane M1—C. albicans+C. glabrata; lane M2—C. albicans+C. krusei; lane M3—C. albicans+C. parapsilosis; lane M4—C. albicans+C. tropicalis; and lane M5—C. albicans+C. glabrata+C. krusei, respectively.

### DETAILED DESCRIPTION OF THE INVENTION

The present invention provides in its most general form, a method to detect and identify in a rapid and precise manner, Candida species with clinical importance in a determined sample, comprising the following steps:
1. (i) Release, isolation and/or concentration of the nucleic acids of the fungi possibly present in the sample,
1. (ii) amplification by multiplex PCR of specific fragments of the ITS regions in the nucleic acids of step (i), according to the Candida species considered,
1. (iii) visualization of the products of the amplification of step (ii) by agarose gel electrophoresis.

The amplification of nucleic acids is performed by a PCR reaction (Saiki et al., Science 239:487-91 (1988)). In the amplification of the step (ii), the following primers are used in the PCR reaction: GTCAAACTTGGTCATTTA (UNI1—Seq ID no. 1), TTCTTTTCCTCCGCTTATTGA (UNI2—Seq ID no. 2), agctgccgccagaggtctaa (Calb—Seq ID no. 3), gatttgcttaattgccccac (Ctro—Seq ID no. 4), gtcaaccgattatttaatag (Cpar—Seq ID no. 5), CTGGCCGAGCGAACTAGACT (ckru—Seq ID no. 6), TTCGGAGCAACGCCTAACCG (Clus—Seq ID no. 7), TTGGCCTAGAGATAGGTTGG (cgui—Seq ID no. 8), CTCAAACCCCTAGGGTTTGG (cdub—Seq ID no. 9). The sequences presented as Seq ID no. 1 and 2 represent the universal primers and their target is the terminal region of the 18S unit and the initial region of the 25S unit, respectively, of all the fungi belonging to the Candida genera. The expression ‘universal primers’ means that this primer set amplifies the ITS-1 and ITS-2 regions in the big majority, or even in the entirety, of fungal species. The sequences of the universal primers are phylogenetically conserved in a way that allows the DNA amplification of different genera of fungi and their targets are the rRNA genes that flank the ITS regions, i.e. the rRNA genes 18 s and 25 s. The universal primers used in the method reported in the present invention were described by Trost et al. (Trost et al., J. Microbiol. Methods 56:201-11 (2004)).

The PCR products resulting from the amplification using the universal primers (SEQ ID NO 1, 2) present sizes ranging from 433 bp (C. lusitaniae) to 929 bp (C. glabrata). However, in this way, the majority of Candida species is not easily discriminated having only with base on these fragments. Therefore, exclusive variations in the ITS-1 and ITS-2 regions of each Candida species were used to design primers with the intent of amplifying a second fragment of minor size, which would facilitate the discrimination of the considered Candida species. In this sense, specific variations in the ITS-1 and ITS-2 regions from the type strains and all the clinical strains available in the database of EMBL/GenBank were analyzed through interspecies alignment to find blocks of conserved regions among different strains. Next, these sequences were compared among species in order to find variable regions that would allow designing specific primers for each Candida species. This in silico study concluded with the design of primers in variable regions among species, but at the same time, conserved among strains (SEQ ID NO: 3-9), excluding by that the possibility of occurring interspecies variability. In the particular case of C. glabrata, a strategy based on a single fragment was designed, since the size of the obtained fragment by amplification with the universal primers (929 bp) allowed the easy discrimination of this species. The sequences presented as Seq ID no. 3, 4, 5, 6, 7, 8 and 9 represent the specific primers for each one of the Candida species, and that have as target, as illustrated next in the Examples section (see Table I): the ITS-1 region from C. albicans (Seq ID no. 3), the ITS-1 region from C. tropicalis (Seq ID no. 4), the ITS-1 region from C. parapsilosis (Seq ID no. 5), the ITS-2 region from C. krusei (Seq ID no. 6), the ITS-2 region from C. lusitaniae (Seq ID no. 7), the ITS-1 region from C. guilliermondii (Seq ID no. 8), and the ITS-2 region from C. dubliniensis (Seq ID no. 9). It is important to note that all the specific primers amplify in the opposite direction of the UNI2 primer, except the Clus primer (Seq ID no. 7), which amplifies in the opposite direction of the UNI1 primer. A single base pair difference is sufficient to design a discriminatory primer. The primers used in this invention and its sequence, and the sizes of the PCR products to obtain are described in Table I.

TABLE I
universal and specific primers for each one of the different Candida
species used in the multiplex PCR methodology according to the present
invention, the respective nucleotide sequence, and the predicted
size of the visualized fragments after agarose gel electrophoresis.

| | Primer | Nucleotide | Size of the fragment |
|---|---|---|---|
| Species | designation | sequence (5′-3′) | (bp) |
| All the fungi | UNI1 | gtcaaacttggtcattta | Trost et al.,(2004) |
| | UNI2 | ttcttttcctccgcttattg | |
| C. albicans | Calb | agctgccgccagaggtctaa | 583/446 |
| C. glabrata | — | — | 929/— |
| C. tropicalis | Ctro | gatttgcttaattgccccac | 583/507 |
| C. parapsilosis | Cpar | gtcaaccgattatttaatag | 570/370 |
| C.. krusei | Ckru | ctggccgagcgaactagact | 590/169 |
| C. lusitaniae | Clus | ttcggagcaacgcctaaccg | 433/329 |
| C. guilliermondii | Cgui | ttggcctagagataggttgg | 668/512 |
| C. dubliniensis | Cdub | ctcaaacccctagggtttgg | 591/217 |

The methodology herein presented was optimized using DNA from Candida type strains and tested in various strains isolated from clinical specimens. Thus, the Candida strains used to validate the method were isolated from clinical specimens in two hospitals in Portugal, one in the north (Hospital de Sao Joao, Porto) and other in the south of the country (Hospital de Santa Maria, Lisboa), in a total of 386 clinical isolates (245 C. albicans, 61 C. parapsilosis, 25 C. tropicalis, 19 C. krusei, 18 C. glabrata, 13 C. guilliermondii and 5 C. lusitaniae) and 8 type strains (C. albicans ATCC 18804, C. glabrata ATCC 2001, C. tropicalis ATCC 750, C. parapsilosis ATCC 22019, C. krusei ATCC 6258, C. lusitaniae ATCC 34449, C. guilliermondii ATCC 6260 and C. dubliniensis ATCC MYA-646). The primary identification of the clinical isolates was carried out in the hospitals using commercial identification systems based on biochemical characteristics and afterwards confirmed using the multiplex PCR strategy according to the presented invention. Furthermore, all the identifications which results were different from those provided by the reference institutions were confirmed using molecular fingerprinting (Correia et al., J. Clin. Microbiol. 42:5899-903 (2004)).

The amplification of nucleic acids to its detection has as an obvious advantage the fact that the detection sensitivity is increased. Furthermore, using universal primers, ITS regions from several Candida species can be amplified conjointly, while the posterior amplification using specific primers allows the identification of multiple species simultaneously present in the same sample. It is important to note that the ITS regions, from which the specific primers were designed, have no DNA sequence match in mammals, bacteria or virus. This is important since the falsely positive amplification of mammal DNA that would be present in the clinical samples is avoided. All the primers used were synthesized by MWG Biotech.

The present invention has further the obvious possibility of preparation of kits containing the necessary elements in order to perform the process. The kit must comprise a compartmentalized transport system in small cells in order to receive in a tight confinement, the necessary reagents. The reagents used in the invention must be provided in the kit in predetermined amounts use in the process of Candida species identification. One or more cells must contain the primer mixture and the remaining reagents, including the Taq polymerase enzyme to be used in the PCR reactions, in the lyophilized form or in an appropriate buffer solution.

### EXAMPLES

### Example 1

### DNA Isolation of Candida Cells in Culture

Candida cells were grown overnight in liquid YEPD medium at 26° C. with aeration on a mechanical stirrer (150 rpm). The cells were collected by centrifugation and the sediment was suspended in 200 μl of lysis buffer (2% Triton X-100, 1% SDS, 100 mM NaCl, 10 mM Tris-Hcl e 1 mM EDTA, pH 8.0). For cellular disrupture, 200 μl of glass beads with a 0.5 mm diameter and 200 μl of phenol/chloroform (1:1) were added and the tubes agitated during three intervals of 60s intercalated with periods of cooling on ice. After the removal of cellular debris by a centrifugation of 5 minutes at 18.000×g, the supernatant was collected and 1 ml of cold absolute alcohol was added before mixing by inversion. The tubes were centrifuged at 18.000×g during 3 minutes and the sediment was suspended in 400 μl of TE buffer (100 mM Tris-HCl, 1 mM EDTA, pH 8.0). A 5 minutes treatment with RNase A (1 mg/ml) at 37° C. was carried out before the addition of 10 μl of sodium acetate 3 M. The DNA was again precipitated by the addition of 1 ml of cold absolute alcohol, mixed by inversion and once again centrifuged. Finally, the DNA was dried at air temperature and suspended in 50 μl of sterilized water. The DNA content and its purity were determined by spectrophotometry at 260 and 280 nm and diluted to a final concentration of 100 ng/μl to be used according to Example 2.

### Example 2

### Multiplex PCR Amplification

Multiplex PCR amplification was performed in a 20 μl volume consisting of 0.8×PCR buffer [160 mM (NH₄)₂SO₄, 670 mM Tris-HCl (pH 8.8)], 3.5 mM MgCl₂, dNTPs mixture (200 μM each), primer mix (SEQ ID NO 1 and 2, 0.55 μM each; SEQ ID 3 and 6, 0.15 μM each; SEQ ID NO 4 and 7, 0.2 μM; SEQ ID NO 5, 0.3 μM; SEQ ID NO 8, 0.05 μM; SEQ ID NO 9, 0.4 μM), 1 U Taq polymerase DNA and 100 ng of genomic DNA, with the remaining volume consisting of sterilized water. To perform multiplex PCR amplification using whole cells, part of an isolated colony was directly suspended in the reaction tube. The reaction was carried out as usual in a thermal cycler Biometra Tpersonal (Whatman Biometra) under the following conditions: 40 cycles of 15 s at 94° C., 30 s at 55° C., and 45 s at 65° C., after an initial period of 10 minutes for denaturation and enzyme activation at 94° C. Negative control reactions were performed simultaneously with each test replacing the DNA by sterilized water in the PCR mixture. A 10 μl aliquot from each of the amplification products was separated by electrophoresis in a 2% agarose gel. The use of ethidium bromide (0.5 μg) allowed the visualization of the DNA fragments with a digital imaging system (Alpha Innotech Corporation) and the identification of the Candida species in question was possible by comparison with a 100 bp DNA ladder (Fermentas).

### Example 3

### Detection and Identification of Candida in Peripheral Blood

In order to determine the detection limit of the method, human peripheral blood was seeded separately with cells from several Candida species, amongst which C. albicans, C. glabrata, C. krusei, C. parapsilosis and C. tropicalis, until concentration of 2.5×10⁵ CFU/ml was obtained. The cell number (CFU/ml) was estimated by hemacytometer counting and confirmed by plating serial dilutions of seeded blood with Candida onto solid medium Sabouraud plates and colony counting forming units after 2 days of incubation at 30° C. The seeded blood was then diluted several times with unseeded blood (concentrations in the range from 2.5×10⁵ to 1.25×10³ CFU/ml) and 200 μl of the diluted samples were exposed to DNA isolation, according to Example 4.

### Example 4

### DNA Isolation from Candida Present in Peripheral Blood

In order to isolate DNA from Candida present in peripheral blood, a method based on heat, detergent and mechanical disruption of Candida cells was used, according to Shin et al. (1997). Thus, to 200 μl of the sample obtained as described in Example 3 were added 800 μl of TXTE buffer (10 mM Tris-HCl, 1 mM EDTA, 1% Triton X-100, pH 8.0) in a sterile centrifuge tube of 1.5 ml. The mixture was then incubated for 10 min at 25° C. to originate lysis of blood cells. Debris resulting from the blood cell reupture was collected by centrifugation at 18,000×g for 8 min. Next, the debris was washed three times with TXTE buffer and the Candida cells were suspended in 300 μl of TXTE buffer and 200 μl of glass beads with a diameter of

0.5 mm

were added. After boiling for 15 minutes in a water bath, the mixture was agitated for 20 minutes using a vortex. Finally, the tubes were centrifuged at 18.000×g for 20 s and the supernatant was used in PCR amplification, as described in Example 2.

### BIBLIOGRAPHY

1. Bikandi, J., R. S. Millan, M. D. Moragues, G. Cebas, M. Clarke, D. C. Coleman, D. J. Sullivan, G. Quindos, and J. Ponton. 1998. Rapid identification of Candida dubliniensis by indirect immunofluorescence based on differential localization of antigens on C. dubliniensis blastospores and Candida albicans germ tubes. J. Clin. Microbiol. 36:2428-2433.
2. Buchman, T. G., Rossier, M., Merz, W. G., Charache, P. 1990. Detection of surgical pathogens by in vitro DNA amplification. Part I. Rapid identification of Candida albicans by in vitro amplification of a fungus-specific gene. Surgery 108:338-347.
3. Chang, H. C., S, N. Leaw, A. H. Huang, T. L. Wu, and T. C. Chang. 2001. Rapid identification of yeasts in positive blood cultures by a multiplex PCR method. J. Clin. Microbiol. 39:3466-3471.
4. Correia, A., P. Sampaio, J. Almeida, and C. Pais. 2004. Study of molecular epidemiology of candidiasis in Portugal by PCR fingerprinting of Candida clinical isolates. J. Clin. Microbiol. 42:5899-5903.
5. Diekema, D. J., S. A. Messer, A. B. Brueggemann, S. L. Coffman, G. V. Doern, L. A. Herwaldt, and M. A. Pfaller. 2002. Epidemiology of candidemia: 3-year results from the emerging infections and the epidemiology of Iowa organisms study. J. Clin. Microbiol. 40:1298-1302.
6. Edmond, M. B., S. E. Wallace, D. K. McClish, M. A. Pfaller, R. N. Jones, and R. P. Wenzel. 1999. Nosocomial bloodstream infections in United States hospitals: a three-year analysis. Clin. Infect. Dis. 29:239-244.
7. Fridkin, S. K. and W. R. Jarvis. 1996. Epidemiology of nosocomial fungal infections. Clin. Microbiol. Rev. 9:499-511.
8. Fujita, S., B. A. Lasker, T. J. Lott, E. Reiss, and C. J. Morrison. 1995. Microtitration plate enzyme immunoassay to detect PCR-amplified DNA from Candida species in blood. J. Clin. Microbiol. 33:962-967.
9. Jarvis, W. R. 1995. Epidemiology of nosocomial fungal infections, with emphasis on Candida species. Clin. Infect. Dis. 20:1526-1530.
10. Kao, A. S., M. E. Brandt, W. R. Pruitt, L. A. Conn, B. A. Perkins, D. S. Stephens, W. S. Baughman, A. L. Reingold, G. A. Rothrock, M. A. Pfaller, R. W. Pinner, and R. A. Hajjeh. 1999. The epidemiology of candidemia in two United States cities: results of a population-based active surveillance. Clin. Infect. Dis. 29:1164-1170.
11. Kiehn T. E., Capitolo C., Mayo J. B., Armstrong D. 1981. Comparative recovery of fungi from biphasic and conventional blood culture media. J. Clin. Microbiol. 14(6):681-683
12. Maaroufi, Y., N. Ahariz, M. Husson, and F. Crokaert. 2004. Comparison of different methods of isolation of DNA of commonly encountered Candida species and its quantitation by using a real-time PCR-based assay. J. Clin. Microbiol. 42:3159-3163.
13. Nguyen, M. H., J. E. Peacock, Jr., A. J. Morris, D. C. Tanner, M. L. Nguyen, D. R. Snydman, M. M. Wagener, M. G. Rinaldi, and V. L. Yu. 1996. The changing face of candidemia: emergence of non-Candida albicans species and antifungal resistance. Am. J. Med. 100:617-623.
14. Panaccio, M. and A. Lew. 1991. PCR based diagnosis in the presence of 8% (v/v) blood. Nucleic Acids Res. 19:1151.
15. Pfaller, M. A. and D. J. Diekema. 2002. Role of sentinel surveillance of candidemia: trends in species distribution and antifungal susceptibility. J. Clin. Microbiol. 40:3551-3557.
16. Roberts G. D., Washington J. A. 1975. Detection of fungi in blood cultures. J. Clin. Microbiol. 1(3):309-310.
17. Saiki, R. K., D. H. Gelfand, S. Stoffel, S. J. Scharf, R. Higuchi, G. T. Horn, K. B. Mullis, and H. A. Erlich. ‘Primer-Directed Enzymatic Amplification of DNA with a Thermostable DNA Polymerase.’ Science 239 (1988): 487-491.
18. Shin, J. H., F. S, Nolte, and C. J. Morrison. 1997. Rapid identification of Candida species in blood cultures by a clinically useful PCR method. J. Clin. Microbiol. 35:1454-1459.
19. Trost, A., B. Graf, J. Eucker, O, Sezer, K. Possinger, U. B. Gobel, and T. Adam. 2004. Identification of clinically relevant yeasts by PCR/RFLP. J. Microbiol. Methods 56:201-211.
20. Williams, D. W., M. J. Wilson, M. A. Lewis, and A. J. Potts. 1995. Identification of Candida species by PCR and restriction fragment length polymorphism analysis of intergenic spacer regions of ribosomal DNA. J. Clin. Microbiol. 33:2476-2479.

1. A DNA fragment isolated from the ITS-1 and ITS-2 regions of Candida, selected from the group consisting of:
a) SEQ ID NO: 3,
b) SEQ ID NO: 4,
c) SEQ ID NO: 5,
d) SEQ ID NO: 6,
e) SEQ ID NO: 7,
f) SEQ ID NO: 8,
g) SEQ ID NO: 9,
and complementary sequences to these.

2. A primer characterized in that it consists in a nucleotide sequence identical to any sequence selected from SEQ ID NOs: 3-9 according to claim 1.

3. A set of primers characterized in that it contains universal primers to fungi and at least one primer according to claim 2

4. A method to amplify DNA fragments with a nucleotide sequence identical to any sequence from SEQ ID NOs: 3-9 according to claim 1, characterized by including the performance of PCR using the set of primers according to claim 3.

5. The method according to claim 4, characterized in that the performed PCR is a multiplex PCR.

6. A method to selectively detect and identify Candida species present in clinical samples, characterized in that it comprises the following steps:
a) amplification by multiplex PCR of DNA from Candida species present in the sample, using the set of primers according to claim 3; and
b) detection of the amplification products.

7. The method according to claim 5, characterized in that the DNA of the Candida species present in the sample is isolated DNA, from whole cells or cell extracts from Candida, or from a polyfungal culture of Candida.

8. The method according to any of the claims 6 or 7, characterized in that the used set of primers comprises the primer with a nucleotide sequence identical to SEQ ID NO: 3 and the identified fungus is from the Candida albicans species.

9. The method according to any of the claims 6 or 7, in which the used set of primers comprises the primer with a nucleotide sequence identical to SEQ ID NO: 4 and the identified fungus is from the Candida tropicalis species.

10. The method according to any of the claim 6 or 7, characterized in that the used set of primers comprises the primer with a nucleotide sequence identical to SEQ ID NO: 5 and the identified fungus is from the Candida parapsilosis species.

11. The method according to any of the claims 6 or 7, characterized in that the used set of primers comprises the primer with a nucleotide sequence identical to SEQ ID NO: 6 and the identified fungus is from the Candida krusei species.

12. The method according to any of the claims 6 or 7, characterized in that the used set of primers comprises the primer with a nucleotide sequence identical to SEQ ID NO: 7 and the identified fungus is from the Candida lusitaniae species.

13. The method according to any of the claims 6 or 7, characterized in that the used set of primers comprises the primer with a nucleotide sequence identical to SEQ ID NO: 8 and the identified fungus is from the Candida guilliermondii species.

14. The method according to any of the claims 6 or 7, characterized in that the used set of primers comprises the primer with a nucleotide sequence identical to SEQ ID NO: 9 and the identified fungus is from the Candida dubliniensis species.

15. A kit to selectively detect and identify Candida species present in clinical samples in compliance with the method according to any one of claims 6 to 14, characterized in that it uses a set of primers according to claim 3.


SEQUENCE LISTING (9 sequences)

SEQ ID NO: 1 - DNA, 18 bp, Candida albicans
gtcaaacttggtcattta

SEQ ID NO: 2 - DNA, 21 bp, Candida albicans
ttcttttcctccgcttattga

SEQ ID NO: 3 - DNA, 20 bp, Candida albicans
agctgccgccagaggtctaa

SEQ ID NO: 4 - DNA, 20 bp, Candida tropicalis
gatttgcttaattgccccac

SEQ ID NO: 5 - DNA, 20 bp, Candida parapsilosis
gtcaaccgattatttaatag

SEQ ID NO: 6 - DNA, 20 bp, Candida krusei
ctggccgagcgaactagact

SEQ ID NO: 7 - DNA, 20 bp, Candida lusitaniae
ttcggagcaacgcctaaccg

SEQ ID NO: 8 - DNA, 20 bp, Candida guilliermondii
ttggcctagagataggttgg

SEQ ID NO: 9 - DNA, 20 bp, Candida dubliniensis
ctcaaacccctagggtttgg